(19) United States
(12) Patent Application Publication
(10) Pub. No.: US 2017/0178540 A1
(43) Pub. Date: Jun. 22, 2017

(54) INJECTION TRAINING WITH MODELED BEHAVIOR

(71) Applicant: TruInject Medical Corp., Irvine, CA (US)

(72) Inventors: Gabrielle A. Rios, Irvine, CA (US); Daniel Bryan Laird Edney, Irvine, CA (US); Cheryl R. Aday, Santa Ana, CA (US); Jonathon Duane Mosqueda, Irvine, CA (US)

(21) Appl. No.: 15/388,326

(22) Filed: Dec. 22, 2016

Related U.S. Application Data
(60) Provisional application No. 62/270,876, filed on Dec. 22, 2015.

(51) Int. Cl. G09B 23/28; A61M 5/31

(52) U.S. Cl. CPC G09B 23/285; A61M 5/31; A61M 2205/332; A61M 2205/3306; A61M 2205/502

(57) ABSTRACT

Various systems and methods are provided for injection training by collecting, processing, analyzing and displaying measured information associated with the delivery of an injection. The injection training systems can include an artificial training and certification head (TCH) with internal sensor and software, syringe(s) with one or more sensors and a wireless transmitter, a computer and/or tablet with image and other processors, and backend servers. Sensor-based measurements of a syringe's position and orientation in three-dimensional space relative to a training apparatus are obtained and processed to provide metrics of a trainee's injection performance. The system can provide one-target or two-target scoring injection training. The system can also provide training for multi-target scoring techniques, including but not limited to fanning, linear threading, cross hatching, serial puncture, serial threading, depot injection, Fern Pattern, cone, Z-Track method, and the like.

### INCORPORATION BY REFERENCE TO ANY PRIORITY APPLICATIONS

Any and all applications for which a foreign or domestic priority claim is identified in the Application Data Sheet as filed with the present application are hereby incorporated by reference in their entirety under 37 CFR 1.57.

This application claims benefit of U.S. Provisional Patent Application No. 62/270,876, filed Dec. 22, 2015, and titled “INJECTION TRAINING WITH MODELED BEHAVIOR,” the entire disclosure of which is hereby incorporated by reference and made part of this specification as if set forth fully herein in its entirety.

### BACKGROUND

The present application relates generally to cosmetic and therapeutic training system, and more specifically to systems, devices, and methods for facial injection training.

A variety of medical injection procedures are often performed in prophylactic, curative, therapeutic, or cosmetic treatments. Injections may be administered in various locations on the body, such as under the conjunctiva, into arteries, bone marrow, the spine, the sternum, the pleural space of the chest region, the peritoneal cavity, joint spaces, and internal organs. Injections can also be helpful in administering medication directly into anatomic locations that are generating pain. These injections may be administered intravenously (through the vein), intramuscularly (into the muscle), intradermally (beneath the skin), subcutaneously (into the fatty layer of skin), or by way of intraperitoneal injections (into the body cavity). Injections can be performed on humans as well as animals. The methods of administering injections typically vary for different procedures and may depend on the substance being injected, the needle size, or the area of injection.

Injections are not limited to treating medical conditions, but may be expanded to treating aesthetic imperfections, restorative cosmetic procedures, procedures for treating migraine, depression, epidurals, orthopedic procedures, self-administered injections, in vitro procedures, or other therapeutic procedures. Many of these procedures are performed through injections of various products into different parts of the body. The aesthetic and therapeutic injection industry comprises two main categories of injectable products: neuromodulators and dermal fillers. The neuromodulator industry commonly utilizes nerve-inhibiting products such as Botox®, Dysport®, and Xeomin®, among others. The dermal filler industry utilizes products administered by providers to patients for orthopedic, cosmetic and therapeutic applications, such as, for example, Juvederm®, Restylane®, Belotero®, Sculptra®, Artefill®, Voluma®, Kybella®, Durolane®, and others. The providers or injectors may include plastic surgeons, facial plastic surgeons, oculoplastic surgeons, dermatologists, orthopedist, primary care givers, psychologist/psychiatrist, nurse practitioners, dentists, and nurses, among others.

One of the problems in the administration of injections is that there is no official certification or training process. Anyone with a minimal medical related license may inject a patient. These “injectors” may include primary care physicians, orthopedist, dentists, veterinarians, nurse practitioners, nurses, physician's assistants, aesthetic spa physicians, therapeutic or the patient for self-administered injections. However, the qualifications and training requirements for injectors vary by country, state, and county. For example, in most states in the United States, the only requirement to inject patients with neuromodulators and/or fillers is a nursing degree or medical degree. This causes major problems with uniformity and expertise in administering injections. The drawbacks resulting from a lack of uniformity in training and expertise are widespread throughout the medical industry. Doctors and practitioners often are not well trained in administering injections for diagnostic, therapeutic, and cosmetic purposes. This lack of training often leads to instances of chronic pain, headaches, bruising, swelling or bleeding in patients.

Current injection training options are classroom-based, with hands-on training performed on live models. The availability of models is limited. Moreover, even when available, live models are limited in the number and type of injections that may be performed on them. The need for live models is restrictive because injectors are unable to be exposed to a wide and diverse range of situations in which to practice. For example, it may be difficult to find live models with different skin tones or densities. This makes the training process less effective because patients often have diverse anatomical features as well as varying prophylactic, curative, therapeutic, or cosmetic needs. Live models are also restrictive because injectors are unable to practice injection methods on internal organs due to health considerations. As a result of these limited training scenarios, individuals seeking treatments involving injections have a much higher risk of being treated by an inexperienced injector. This may result in low patient satisfaction with the results, or in failed procedures. In many instances, patients have experienced lumpiness from incorrect dermal filler injections. Some failed procedures may result in irreversible problems and permanent damage to a patient's body. For example, patients have experienced vision loss, direct injury to the globe of the eye, and brain infarctions where injectors have incorrectly performed dermal filler procedures. Other examples of side effects include inflammatory granuloma, skin necrosis, endophthalmitis, injectable-related vascular compromise, cellulitis, biofilm formation, subcutaneous nodules, fibrotic nodules, other infections, and death.

### SUMMARY

Sensor-based injection training systems and methods are described in U.S. patent application Ser. No. 14/645,997, filed on Mar. 12, 2015, and titled “AUTOMATED DETECTION OF PERFORMANCE CHARACTERISTICS IN AN INJECTION TRAINING SYSTEM,” the entire disclosure of which is hereby incorporated by reference and made part of this specification as if set forth fully herein in its entirety.. Systems and methods can be provided for injection training by collecting, processing, analyzing and displaying measured information associated with the delivery of an injection. Sensor-based measurements of a syringe's position and orientation in three-dimensional space are obtained and processed to provide metrics of a trainee's injection performance. The measurements can be combined with a digital model of a training apparatus to deliver a computer-generated, graphical depiction of the training injection, enabling visualization of the injection from perspectives unavailable in the physical world. The training injection execution, as reflected in the measured sensor-based data, can be reviewed and analyzed at times after, and in locations different than, the time and location of the training injection. Additionally, injection training data associated with multiple training injections can be recorded, aggregated and analyzed for, among other things, trends in performance.

In addition, for some injection trainings, the angle and depth of entry of the syringe needle into the training apparatus is of particular importance. For example, to ensure that fillers are injected at the right depth and/or orientation. 9-axis inertia motion sensors (IMS) on the syringe and/or sensors on the training apparatus can be used to measure the position and orientation of the syringe needle to provide graphical depiction of the training injection. However, the graphical depiction still may not provide a direct visual guide to the trainee about where and at which angle the needle should eventually land underneath the skin. In some embodiments described herein, a system can be configured to provide injection training using a two-target scoring system. The two- target scoring system provides two target areas for the trainee, namely an injection entry target on the skin of the training apparatus and a product injection target under the skin. By practicing hitting both targets and following instructions associated with the two-target scoring training, the trainee can have a visual guide, and gradually develop muscle memory for, among other things, the desired angle and depth of needle entry, and/or the injection pressure and duration.

In some embodiments described herein, a system can be configured to provide training of specific injection techniques, including but not limited multi-target scoring techniques. For example, the system described herein can be configured to provide training for a fanning technique. In another example, the system described herein can be configured to provide training for a linear threading technique. For both fanning and linear threading techniques, the system can provide one injection entry target and a plurality of product injection targets. By practicing hitting all the targets and following instructions associated with the fanning or linear threading technique training, the trainee can have a visual guide, and gradually develop muscle memory for, among other things, the desired angle and depth of needle entry, and/or the injection pressure and duration at each product target, and/or the desired depth for needle retraction before moving to a new product target. Additional techniques include but are not limited to cross hatching, serial puncture, serial threading, depot injection, Fern Pattern, cone, Z-Track method, and the like.

The systems, methods, devices, and non-transitory, computer-readable media discussed herein include several aspects, no single one of which is solely responsible for its desirable attributes. Without limiting the scope of the disclosed invention as expressed by the claims which follow, some features are discussed briefly below. After considering this discussion, and particularly after reading the section entitled “Detailed Description,” it will be understood how advantageous features of this disclosure include, among other things, improved injection training.

In one aspect of the present disclosure, an injection training system is described. The injection training system can include a training apparatus configured to receive a training injection, the training apparatus comprising one or more training apparatus sensors; a syringe configured to deliver the training injection, the syringe including a needle and one or more syringe sensors; one or more processors in communication with the training apparatus and the syringe; and a user interface in communication with the one or more processors; wherein when a trainee performs the training injection on the training apparatus using the syringe, the one or more training apparatus and syringe sensors are configured to measure position and/or orientation information of the syringe and the training apparatus, and the one or more processors are configured to receive and process inputs from the one or more training apparatus and syringe sensors so as to output on the user interface three-dimensional digital models of the training apparatus and the syringe corresponding to the position and/or orientation of the syringe relative to the training apparatus as the trainee is performing the training injection. The one or more training apparatus and syringe sensors of the system can be configured to measure one or more of injection force, injection duration, or injection volume, and the one or more processors are configured to receive and process inputs from the one or more training apparatus and syringe sensors so as to output feedback of the one or more of injection force, injection duration, or injection volume, and/or an outcome simulation.

The system can further comprise one or more training modules in communication with the one or more processors, the one or more training modules comprising instructions to a trainee performing the training injection, the instructions configured to be displayed on the user interface. The one or more training modules can comprise a two-target scoring training module, wherein the two-target scoring training module is configured to provide an injection entry target on a surface of the training apparatus and a product injection target under the surface of the training apparatus for display on the user interface. The injection entry target and product injection target can be configured to be displayed sequentially to trace movements of the needle during the training injection. The one or more training modules can comprise a multi-target scoring training module, wherein the multi-target scoring training module is configured to provide an injection entry target on a surface of the training apparatus and a plurality of product injection targets under the surface of the training apparatus for display on the user interface. The plurality of product injection targets can project outward from the injection entry target, and the injection entry target and plurality of product injection targets are configured to be displayed sequentially to trace movements of the needle during the training injection, the injection entry target configured to be displayed first. The plurality of product injection targets can be substantially linearly spaced from the injection entry target and from one another, and the injection entry target and plurality of product injection targets are configured to be displayed sequentially to trace movements of the needle during the training injection, the injection entry target configured to be displayed first and followed by a product injection target farthest from the injection entry target. The multi-target scoring training module can comprise a plurality of injection entry targets.

The one or more processors of the system can be configured evaluate the training injection based on the inputs from the one or more training apparatus and syringe sensors and evaluation logics from one or more training modules. The one or more training apparatus sensors can comprise an optical sensor. The one or more syringe sensors can comprise 9-axis inertial motion sensors. The one or more syringe sensors can comprise a force sensor configured to measure injection force. The syringe can comprise a light emission source. The one or more processors can be configured to record and transmit data associated with the training injection to a database. The data can be configured for playback. The training apparatus can be an artificial human head.

In another aspect of the present disclosure, an injection training system is described. The injection training system can include one or more processors in communication with and receiving inputs from one or more sensors on a training apparatus and/or on a needle; and a user interface in communication with the one or more processors and configured to provide injection instructions associated with the injection technique, the injection instructions including an injection entry target on the training apparatus where the needle should penetrate the training apparatus, and a product injection target on the training apparatus where the training injection should be delivered, wherein the inputs from the one or more sensors on the training apparatus and/or needle comprise injection information associated with the training injection, the injection information including a needle entry location for the training injection, and a product injection location where the training injection was delivered, and wherein the user interface is configured to display indications of positions and/or orientations of the needle and training apparatus, and the one or more processors are configured to analyze the injection information including determining whether the training injection was delivered to the injection entry target and product injection target. The inputs from the one or more sensors on the training apparatus and/or needle can further comprise one or more of an injection force or injection duration. The one or more processors can be configured to evaluate the injection information relative to one or more of the following evaluation criteria: a targeting accuracy score; a depth of the training injection; a duration of the training injection; an angle of entry of the training injection; an injection force; or an amount of therapeutic agent delivered by the training injection.

In another aspect of the present disclosure, an injection training system is described. The injection training system can include one or more processors in communication with and receiving inputs from one or more sensors on a training apparatus and/or on a needle; and a user interface in communication with the one or more processors and configured to provide injection instructions associated with the injection technique, the injection instructions including an injection entry target on the training apparatus where the needle should penetrate the training apparatus, and a plurality of product injection targets on the training apparatus where a plurality of injections should be delivered after the needle penetrates the training apparatus, wherein the inputs from the one or more sensors on the training apparatus and/or needle comprise injection information associated with the training injection, the injection information including a needle entry location for the training injection, and a plurality of product injection locations on the training apparatus where the plurality of injections were delivered during the training injection, and wherein the user interface is configured to display indications of positions and/or orientations of the needle and training apparatus, and the one or more processors are configured to analyze the injection information including determining whether the training injection was delivered to the injection entry target and plurality of product injection targets. The injection instructions can comprise a plurality of injection entry targets and the injection information can comprise a plurality of needle entry locations. The plurality of product injection targets can project outward from the injection entry target and the injection instructions include a needle retracting target near the injection entry target under a surface of the training apparatus after each of the plurality of injections. The plurality of product injection targets can be substantially linearly spaced from the injection entry target and from one another, and the training instructions include displaying sequentially the plurality of product injection targets with decreasing distance from the injection entry target. The inputs from the one or more sensors on the training apparatus and/or needle can further comprise one or more of an injection force or injection duration. The one or more processors can be configured to evaluate the injection information relative to one or more of the following evaluation criteria: a targeting accuracy score; a depth of the training injection; a duration of the training injection; an angle of entry of the training injection; an injection force; a maximum number of injections during the training injection; or an amount of therapeutic agent delivered by the training injection. The training technique can be one of fanning, linear threading, cross hatching, serial puncture, serial threading, depot injection, Fern Pattern, cone, or Z-Track method.

In another aspect of the present disclosure, a method of providing injection training is described. The method can include providing injection instructions associated with the injection technique, the injection instructions including an injection entry target on a training apparatus where a needle should penetrate the training apparatus, and a product injection target on the training apparatus where the training injection should be delivered; obtaining injection information associated with the training injection, wherein the injection information can comprise a needle entry location for the training injection, and a product injection location where the training injection was delivered; displaying on a user interface indications of positions and/or orientations of the needle and training apparatus; and analyzing the injection information including determining whether the training injection was delivered to the injection entry target and product injection target. The plurality of product injection targets can be substantially linearly spaced from the injection entry target and from one another, and the training instructions include displaying sequentially the plurality of product injection targets with decreasing distance from the injection entry target. The one or more processors can be configured to evaluate the injection information relative to one or more of the following evaluation criteria: a targeting accuracy score; a depth of the training injection; a duration of the training injection; an angle of entry of the training injection; an injection force; a maximum number of injections during the training injection; or an amount of therapeutic agent delivered by the training injection.

In yet another aspect of the present disclosure, a method of providing injection training is described. The method can include providing injection instructions associated with the injection technique, the injection instructions including an injection entry target on a training apparatus where a needle should penetrate the training apparatus, and a plurality of product injection targets on the training apparatus where a plurality of injections should be delivered after the needle penetrates the training apparatus; obtaining injection information associated with the training injection, wherein the injection information can comprise a needle entry location for the training injection, and a plurality of product injection locations on the training apparatus where the plurality of injections were delivered during the training injection; displaying on a user interface indications of positions and/or orientations of the needle and training apparatus; and analyzing the injection information including determining whether the training injection was delivered to the injection entry target and plurality of product injection targets. The injection instructions can comprise a plurality of injection entry targets and the injection information can comprise a plurality of needle entry locations. The plurality of product injection targets can project outward from the injection entry target and the injection instructions include a needle retracting target near the injection entry target under a surface of the training apparatus after each of the plurality of injections. The plurality of product injection targets can be substantially linearly spaced from the injection entry target and from one another, and the training instructions include displaying sequentially the plurality of product injection targets with decreasing distance from the injection entry target. The one or more processors can be configured to evaluate the injection information relative to one or more of the following evaluation criteria: a targeting accuracy score; a depth of the training injection; a duration of the training injection; an angle of entry of the training injection; an injection force; a maximum number of injections during the training injection; or an amount of therapeutic agent delivered by the training injection.

For purposes of summarizing the disclosure, certain aspects, advantages, and novel features have been described herein. Of course, it is to be understood that not necessarily all such aspects, advantages, or features will be embodied in any particular embodiment.

Described in further detail below are aspects of systems, methods, devices, and non-transitory computer-readable media for injection training. The aspects may be combined, in whole or in part, with injection training systems such as those from TruInject™ Medical Corporation, assignee of the present application. While reference to TruInject™ Medical Corporation may be included in the description that follows, it will be understood that the aspects described herein may be applied in other injection contexts and systems without departing from the scope of the present disclosure.

### BRIEF DESCRIPTION OF THE DRAWINGS

FIG. 1 illustrates a system diagram of an example injection training system.

FIGS. 2A-E illustrate example displays of various views of a simulated head on a user interface of an example injection training system.

FIGS. 3A-3C illustrate interactions between a trainee using an example injection training system, and user interface and data capturing of the example injection training system.

FIGS. 4A-C illustrate example screenshots of a user interface of an example injection training system during an injection training.

FIG. 5 shows a process flow diagram for a one-target scoring method of providing injection training according to an embodiment of the present disclosure.

FIG. 6 shows a process flow diagram for a two-target scoring method of providing injection training according to an embodiment of the present disclosure.

FIG. 7 shows a process flow diagram for a multi-target scoring method of providing injection training according to an embodiment of the present disclosure.

While the foregoing “Brief Description of the Drawings” references generally various embodiments of the disclosure, an artisan will recognize from the disclosure herein that such embodiments are not mutually exclusive. Rather, the artisan would recognize a myriad of combinations of some or all of such embodiments.

### DETAILED DESCRIPTION

The present disclosure generally relates to injection training systems, methods, devices, and non-transitory, computer-readable media. The system can include a training apparatus with internal sensor(s) and/or software, syringe(s) with one or more sensors and a wireless transmitter, a computer and/or tablet with image and other processors, software and/or backend servers. In some embodiments, the system can further include a stand, a transportation case, charging stations, and accompanying accessories. The training apparatus can be an artificial training head, or any portion of a patient's anatomy, including but not limited to neck, arms, legs, back torso, and the like. In some embodiments, the system can be used with live people. Although the present disclosure is described mainly with respect to a head or face structure, it is to be understood that the present disclosure is not limited to a head or face, but extends to any anatomical structure. By way of non-limiting example, a trainee may practice injecting into a synthetic face of the training head using one of the syringes. A local display device or user interface, which can be an integral part of the computer and/or tablet, and/or a separate device, is configured to show the 3-dimensional position and orientation of the needle tip reflecting information sent from the syringe and/or the training head. Testing performance data can include training pass/fail and scoring information, and can also include information detailing the profile of the syringe interaction with the training head, which can be transmitted from the syringe and/or TCH to the training server. Injection training may include providing a website or cloud-based database accessible by the trainee, health care provider, training company, or injection manufacturer.

### System Overview

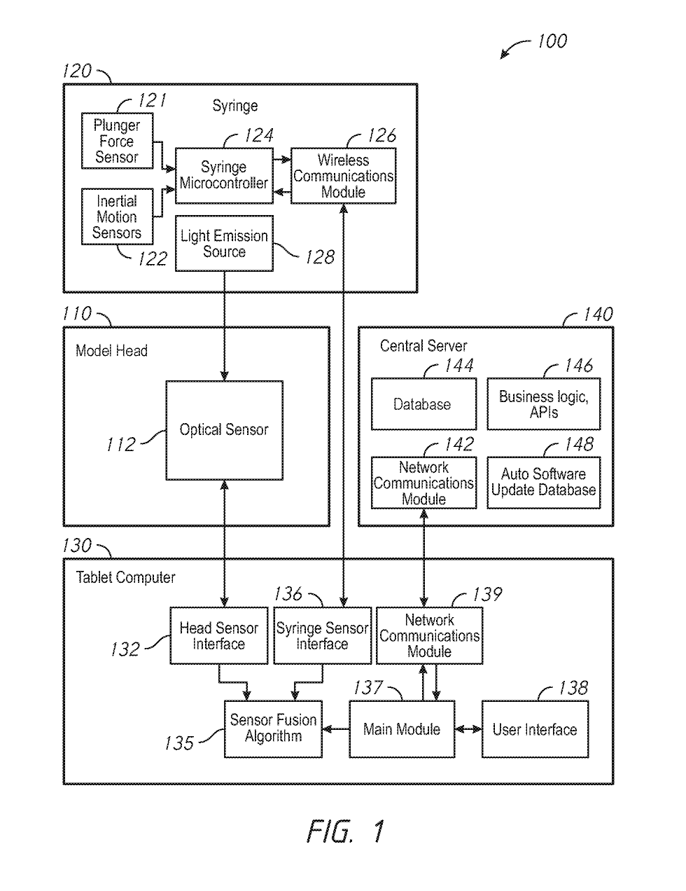

FIG. 1 shows a diagram of an example embodiment of an injection training system 100. The injection training system 100 includes a TCH 110 that is configured to receive a training injection delivered by a syringe 120. The syringe 120 can include a needle having a needle tip at the distal end of the syringe, a barrel, a plunger at a proximal end of the syringe and configured to exert a force on the barrel. The syringe 120 can include one or more syringe sensors, configured to obtain syringe sensor information associated with one or more characteristics of the syringe as it is being used to deliver a training injection. Examples of the sensors include but are not limited to a plunger force sensor 121, IMS 122, a gyroscope, an accelerometer, optical path sensor(s), and the like. Additional details of the syringe sensors and their functions are described in U.S. patent application Ser. No. 15/299,209, filed on Oct. 20, 2016, and titled “INJECTION SYSTEM,” the entire disclosure of which is hereby incorporated by reference and made part of this specification as if set forth fully herein in its entirety..

The syringe 120 can include a syringe processor 124 configured to receive inputs from the one or more syringe sensors, and a wireless communication module or transmitter 126 in communication with the syringe processor 124. The wireless communication module 126 can enable wireless communication via a network with a syringe sensor interface 136 in a local tablet computer 130. Using a tablet computer 130 can advantageously improve portability of the injection training system 100. The network may be a public network including but not limited to the Internet, or a private network including but not limited to a virtual private network, also referred to as a “VPN”. The messages communicated via the network can be transmitted and/or received using appropriate and known network transmission protocols including but not limited to TCP/IP. The network can include one or more networking technologies, such as satellite, LAN, WAN, cellular, peer-to-peer, and the like. In some embodiments, the wireless communication can be via Bluetooth technology. The wireless communication module 126 can provide syringe sensor inputs received by the syringe processor 124 to the syringe sensor interface 136. The syringe 120 can further include a light emission source 128, such as an LED.

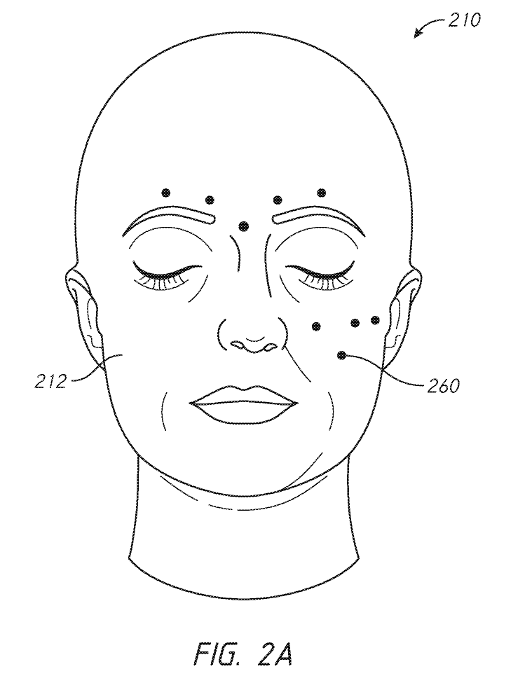

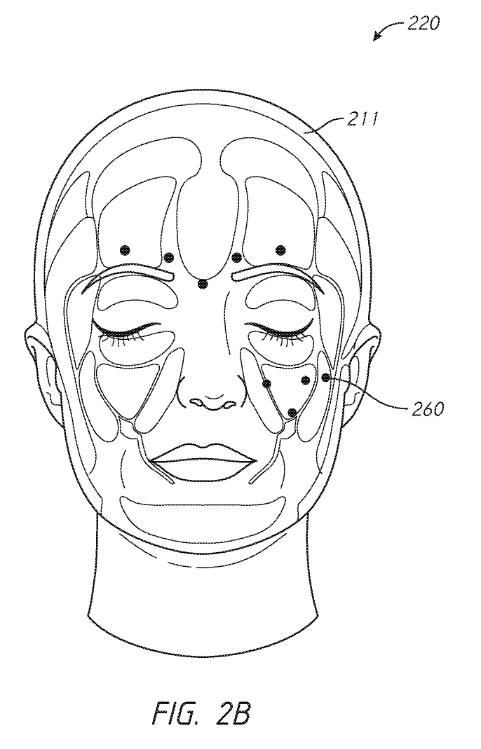

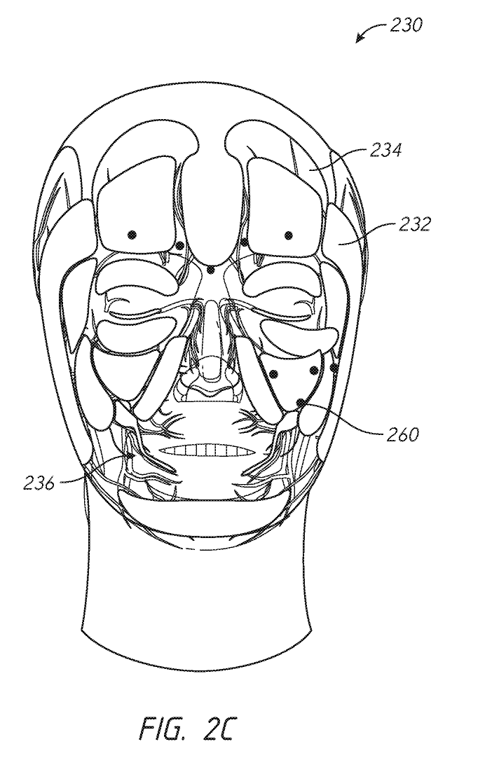

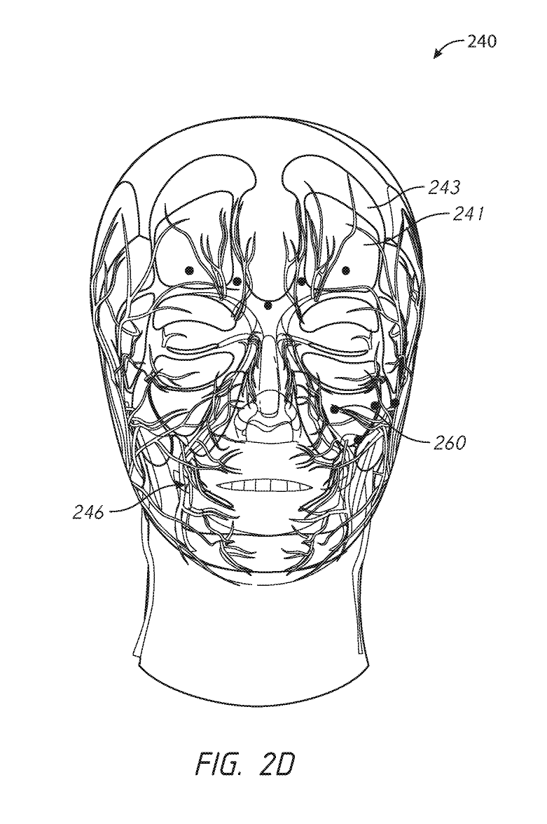

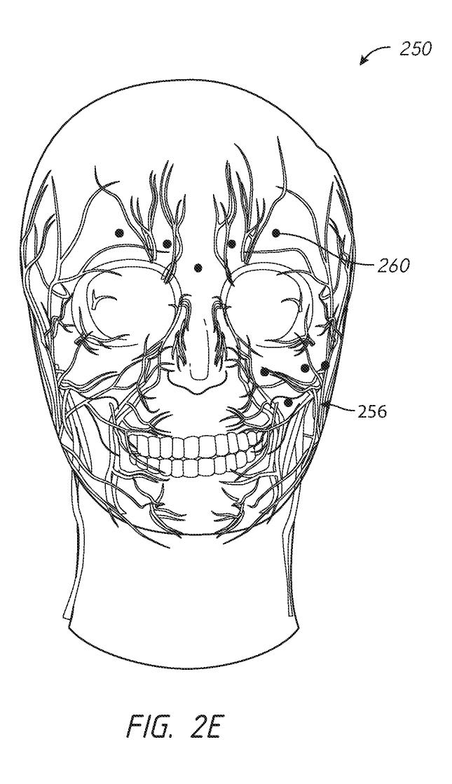

With continued reference to FIG. 1, the training head 110 is configured to represent a human head and face, such as shown in FIGS. 2A-E, which will be described in more detail below. The training head 110 can include internal sensors, such as an optical sensor 112 having one or more image sensors. The optical sensor 112 can detect light signals from the light emission source 128 of the syringe 110. The optical sensor 112 can be configured to communicate via a network with a tablet computer 130. For example, inputs from the optical sensor 112 can be provided to a TCH sensor interface 132 in the tablet computer 130. In some embodiments, the optical sensor 112 can communicate directly with the training head sensor interface 132 via a USB connection. The training head sensor interface 132 can include an image processing algorithm to process the image sensor inputs. Details of the image processing algorithm are described in the '997 Application, referenced herein.

Outputs of the training head sensor interface 132 and the syringe sensor interface 136 can be fed into a sensor fusion algorithm 135 in the tablet computer 130 for determining a position, orientation and/or motion of the syringe 120 relative to the training head 110. Alternatively, processing can occur in the training head or syringe with only final position values sent to the tablet 130. The processed position and motion information can be fed into a main module 137 of the tablet computer 130. The main module 137 can run the training, visualization, and/or administration algorithm of the system 100. For example, the main module 137 can run a tablet computer application. The tablet application can allow the trainee to do training injection for specific products, take exams and get certified, view certifications, purchase products, and/or see their ranking against other trainees. The application can also include a Patient Education section which allows the practitioner trainee to hand the tablet to a patient to view videos about available products and procedures.

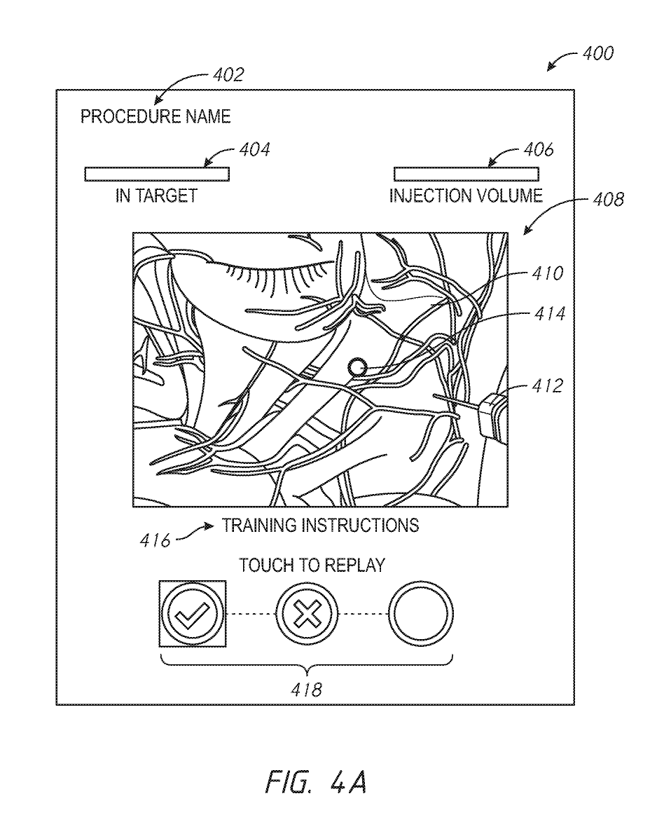

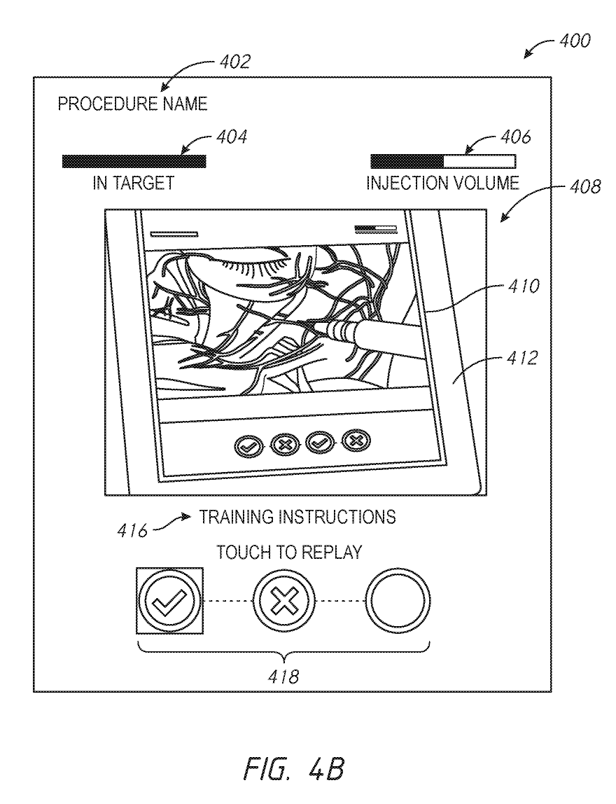

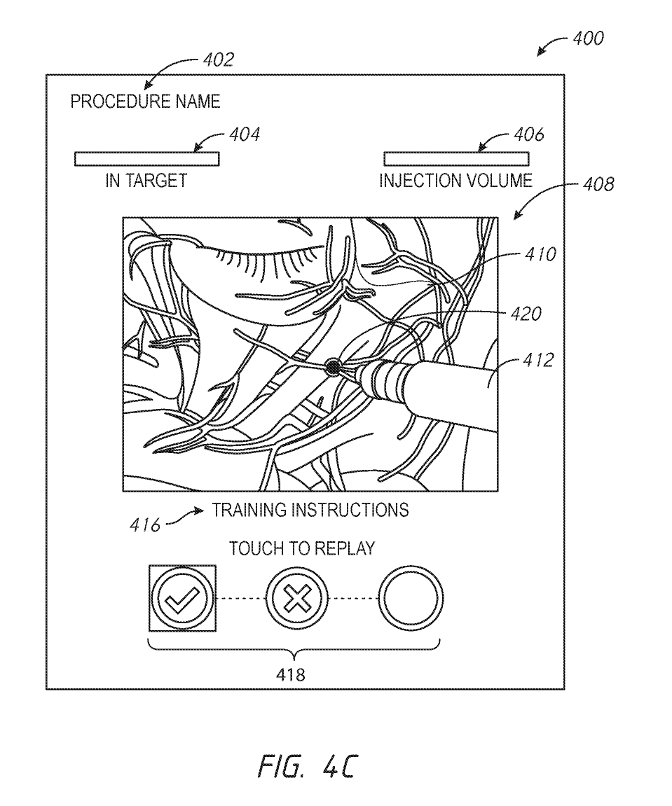

The training portion of the tablet application can be in the form of a training video followed by a series of practice screens (such as those show in FIGS. 4A-C). Each trainee completes a set of training injections with the proper level of accuracy (as determined by scoring) before that practitioner can take the certification exam. Live feedback on method of injection can be available by sound, visual, or other cues. Trainees can replay their injections during training to better understand their sources. Once a trainee has completed training with a passing score, the trainee may attempt certification via an exam.

In some embodiments, the main module 137 can run a web application. The web application can be similar to the tablet-based application. For example, this web application can allow the trainee to view certifications, purchase products, and/or see his or her ranking against other practitioners, and/or to download new techniques.

The tablet computer 130 can include a built-in display or user interface 138, or be connected to a separate user interface device, in communication with the main module 137. The user interface 138 can include a visual display such as a touchscreen, monitor, display screen, or the like. In some embodiments, the visual display may also be used as an input device, such as a touchscreen. In some embodiments, an input device such as a keyboard, keypad, mouse, stylus, camera, biometric sensor, or the like may be coupled with the user interface 138 and configured to provide messages to the user interface 138.

The tablet computer 130 can also communicate directly or via the network with network communication modules 139, 142 of the tablet computer 130 and a central training server 140. The training server 140 can have access to database 144 that stores information related to use of the training system 100, such as injection records, exam results, and/or product data. The database 144 may be configured to maintain records for trainees, exercise performance information, observation notes, and other training related data. The database 144 may also be configured to store training content such as the exercises, educational materials, instructional content, curricula organizing the instructional content for trainees, and the like. The database 144 may be in direct communication with the training server 140 or may be accessible via the network. The training server 140 can further include an auto-update software 148 to update the database 144.

The training server 140 can further include training and examination business logic and application program interfaces 146 with scoring methods and processes. The training server 140 may provide information about a particular trainee via a training portal. The training portal may also be configured to provide a representation of the information about a particular trainee and/or aggregated trainee information to third parties, such as a healthcare provider or a company administering a training center where the training head 110 is located. The injection training system 100 can further be configured to allow a super user account to collect cross-practice data. The injection training system 100 can allow a practice administrator to set up new trainees and/or purchase new products. For example, each practice administrator may be able to set up three trainees per system. The injection training system 100 can allow a company administrator to purchase systems, request service, create company sales representatives, assign training equipment to sales representatives, and/or select the company product list for a company that makes a product that can be certified using the injection training system 100. The injection training system 100 can allow sales representatives be assigned training equipment and practices, and be responsible for managing distribution of training equipment to each practice.

Additional details of the injection training system as illustrated in FIG. 1 are described in the '997 Application, which is referenced herein.

### User Interface

FIGS. 2A-E shows various views of a simulated human head 210, 220, 230, 240, 250 that can be selectively displayed to a trainee on the user interface of the injection training system. The simulated human head 210, 220, 230, 240, 250 can be a digital model of the training head 110 by a 3D scene renderer. FIGS. 2A-E illustrate the digital model displaying different information corresponding to different anatomical structures. FIG. 2A illustrates the simulated head 210 with an opaque skin layer 212. FIG. 2B illustrates the simulated head 220 with a transparent skin layer 211 and all the layers that under the skin layer 211 are visible. FIG. 2C illustrates the simulated head 230 with no skin layer, but with fat pads 232, muscles 234, and nerves and blood vessels 236. FIG. 2D illustrates the simulated head 240 with no skin layer, transparent fat pads 241 and transparent muscles 243, and opaque nerves and blood vessels 246. FIG. 2E illustrates the simulated head 250 displaying only the nerves and blood vessels 256. In all FIGS. 2A-E, markers 260 are shown on various landmarks on the face of the simulated head 210, 220, 230, 240, 250. In some embodiments, these markers 260 represent injection targets. In some embodiments, different layers of the anatomical structure can be shown in different colors to distinguish the layers and/or tissue structures.

During an injection the user interface can graphically illustrate the different anatomical skin, tissue, and vasculature layers as the needle tip penetrates each layer. For example, this can be accomplished by graphically revealing different anatomical layers as the layers are penetrated by the needle. The different anatomical layers can be labeled or provided with different textures or colors to indicate a new layer is shown. In an embodiment, the trainee can navigate between the graphical representations of the different anatomical layers, such as those illustrated in FIGS. 2A-E, as desired. For example, the trainee can swipe up or down to remove layers from the simulated head. In some embodiments, the trainee can use two or more fingers to move the simulated head on the screen (“Pan” function). In some embodiments, the trainee can move two fingers apart to zoom into a particular portion of the simulated head and use pinching movements of two fingers to zoom out. In some embodiments, the trainer can rotate the simulated head by using a finger to turn the head from side to side. The pan, zoom, and rotate functions allow the trainee to better visualize the injection as it is being performed or afterwards. The user interface can be used with the HoloLens technology, including but not limited to air-tap gestures by the trainee.

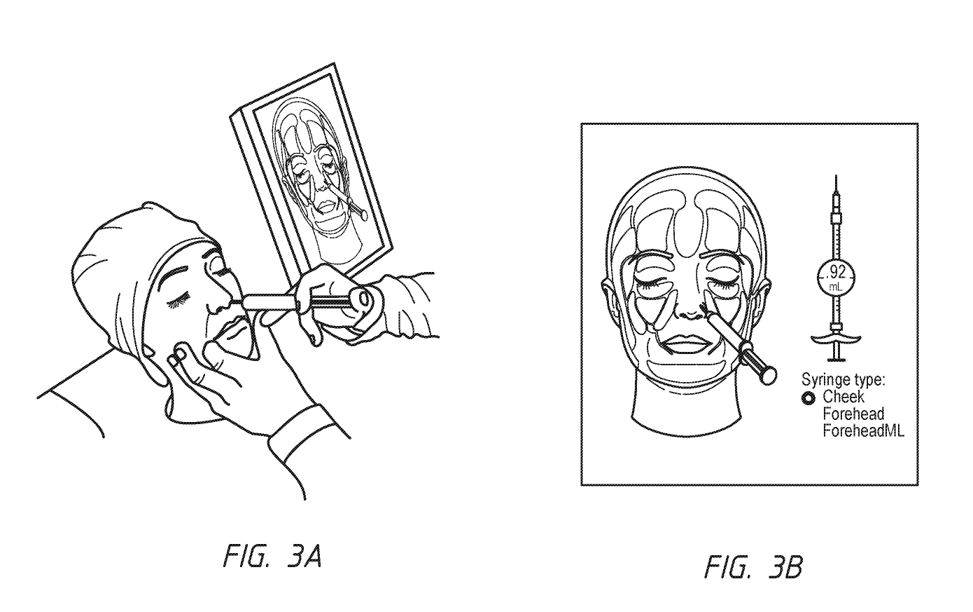

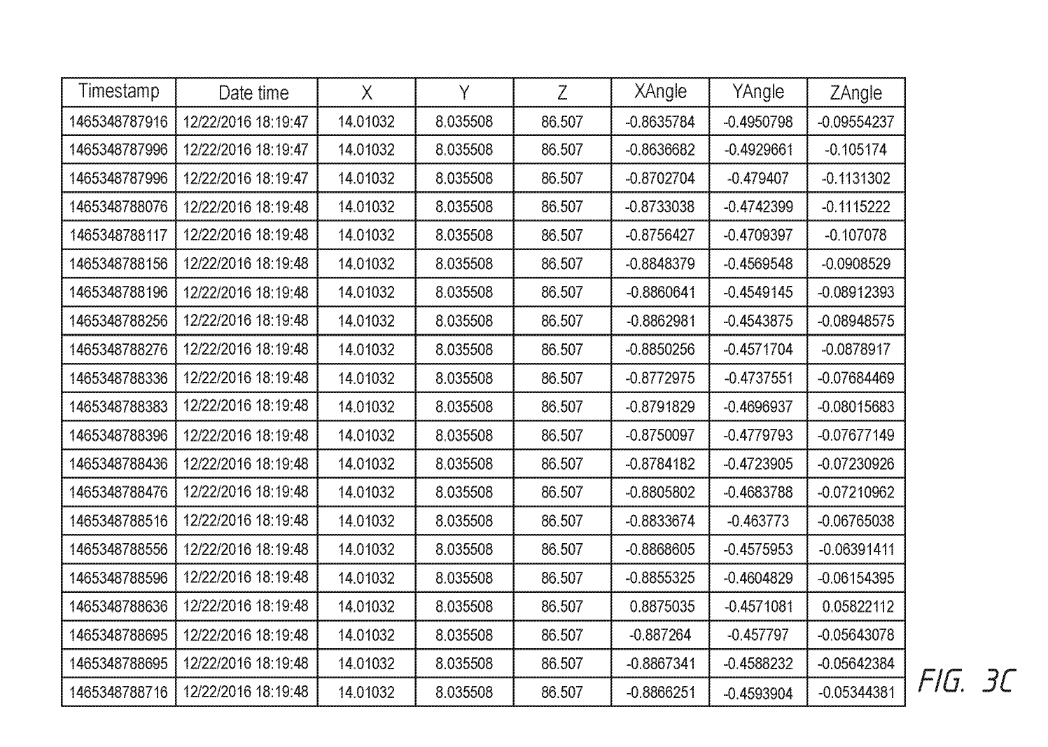

The real-time graphical depiction of the training injection, as presented on the user interface, provides information to help the trainee effectively deliver the desired injection. As shown in FIGS. 3A-C, as the trainee is performing a training injection (FIG. 3A), the user interface can display simulation of the syringe needle and/or the simulated head as described above (FIG. 3B). One of skill will appreciate that the syringe shown in FIGS. 3A-B can be any type of syringes described herein or known in the art. The user interface can further display performance indicators for the training injection. The trainee may alter the view of the graphical depiction to provide different perspectives of the training injection as it is being performed. For example, the trainee might select to view on the user interface vital structures that must be avoided. The training system can compare the position of the syringe needle tip, based on the determined position and orientation of the syringe, to the desired target, such as the markers 260, and to vital structures as the basis for evaluating the skill of the trainee. Further, the training system can capture and record all the data related to the injection training procedure (FIG. 3C). The captured data can be used for record keeping, certification, review, replay, and other purposes. Injection training data associated with multiple training injections of a single trainee can be aggregated and analyzed for, among other things, trends in performance. In some embodiments, training data associated with different trainees can be aggregated and analyzed.

FIGS. 4A-D illustrate example screenshots of the user interface 400 during an example training injection. As illustrated in FIG. 4A, the user interface 400 displays a procedure name 402 of the current injection training procedure. The user interface 400 can further display an indication of whether the syringe needle is in the target or how close the syringe needle is to the center of the target, such as scores, text messages, or an “IN TARGET” bar 404. The user interface 400 can also display an indication of the amount of medication or product injected, such as numbers, percentages, text messages, or an “INJECTION VOLUME” bar 406. The “IN TARGET” bar 404 and “INJECTION VOLUME” bar 406 are both empty in FIG. 4A as the needle has not made contact with the training head, as shown in the graphic 408 on the user interface 400. The graphic 408 of FIG. 4A displays a zoomed-in image of the simulated head 410 and a digital model of the syringe needle 412. The graphic 408 can further display an injection target 414. In some embodiments, the injection target 414 can be in the form of a green circle or sphere. Other forms of injection target can be used. The user interface 400 can include training instructions 416 and information about previous and current training injections, for example, in the form of circles 418 with a tick indicating successful past training injection, an “x” indicating failed past training injection, and an empty circle indicating the current training injection. In some embodiments, the user interface 400 can allow the trainee to replay past procedures by touching the circles indicating past procedures. A skilled artisan will appreciate that locations of various components of the user interface as shown in FIGS. 4A-C and described herein are for illustrating purposes and non-limiting.

Turning to FIG. 4B, the graphic 408 indicates that the trainee's syringe needle has penetrated the skin of the training head by showing that the needle tip of the simulated needle 412 is underneath the transparent skin layer of the simulated head 410. The target 414 of FIG. 4A is no longer in the graphic 408 as the system determines that the needle is within the target. In some embodiments, the target can continue to be displayed throughout the training injection. The user interface 400 shows a full “IN TARGET” bar 404. In some embodiments, a full “IN TARGET” bar 404 indicates that the needle is very close to or at the center of the injection target. The user interface 400 also shows a half-full “INJECTION VOLUME” bar 406, indicating that the trainee is still injecting medication or product into the training head. When the system determines that the desired injection volume has been reached, the “INJECTION VOLUME” bar 406 can become a full bar. Thereafter when the needle is retracted from the training head, the blank circle among the circles 418 can turn into a circle with a tick.

Turning to FIG. 4C, the graphic 408 indicates that the trainee's syringe needle has penetrated the skin of the training head by showing that the needle tip of the simulated needle 412 is underneath the transparent skin layer of the simulated head 410. However, although the target 414 of FIG. 4A is no longer in the graphic 408, a warning icon 420, such as a red circle or sphere, appears in the graphic 408 as the system determines that the needle has missed the target, and/or the needle has hit a nerve or blood vessel. The system can stop recording any sensor inputs once the system determines that the needle is not within the target. As a result, both the “IN TARGET” bar 404 and INJECTION VOLUME″ bar 406 remain empty. Further, the blank circle among the circles 418 can turn into a circle with an “x”.

### Examples of Injection Trainings

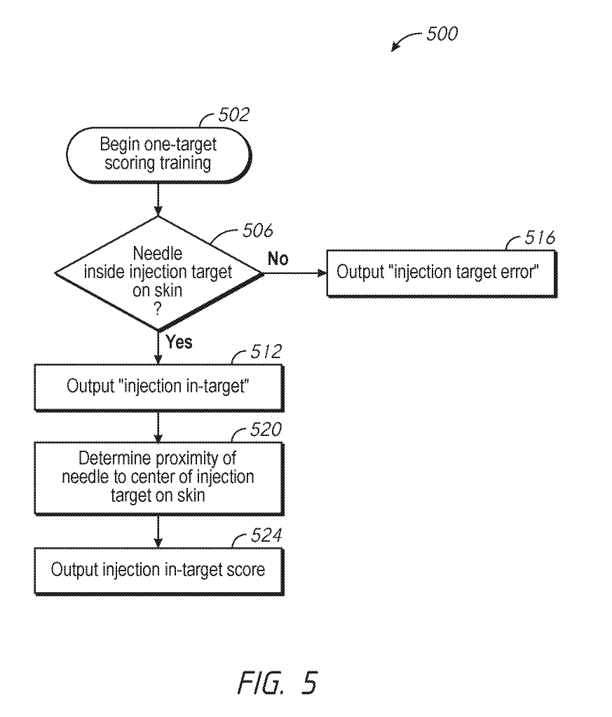

Examples of various injection training methods will now be described with reference to FIGS. 5-7. FIG. 5 is a process flow diagram for a method of providing a one-target scoring training 500. The method shown in FIG. 5 may be performed in one or more of the devices shown herein.

The method 500 begins at node 502 where a trainee begins the one-target scoring training. In some embodiments, the trainee begins the one-targeting scoring training by pushing a corresponding button on the user interface.

At decision node 506, the system determines if the syringe needle is within an injection target. In some embodiments, the injection target can be an entry target on the skin. In some embodiments, the injection target can be the actual product or medication injection target underneath the skin. In some embodiments, the target can be a circle. In some embodiments, the target can be a sphere in the 3D-rendered simulated head. If the needle is outside the target as determined by the sensors in the training head, the system can output and display on the user interface an “Injection Target Error” message or an equivalent indication, such as shown in FIGS. 4A-C at node 516. If the needle is within the target, the system can output and display on the user interface an “Injection In-Target” message or an equivalent indication, such as shown in FIGS. 4A-C at node 512. Optionally, the system can further determine how close the needle is to the center of the target at node 520. The system can further optionally output and display on the user interface an injection in-target score at node 524. For example, the injection in-target score can be presented as a numerical value, a grade, percentage, or how full the “IN TARGET” bar is. Example steps of the one-target scoring training are provided in Table 1 below.

TABLE 1

| State Name | Action/Outcome | Next State |
|---|---|---|
| SyringeWaiting | Syringe becomes visible to | InjectionCorrect |
| | sensor, is inside of injection | |
| | target area on skin | |
| SyringeWaiting | Syringe enters skin, is visible | InjectionWrong |
| | outside of injection target area | |
| InjectionCorrect | Injection Item Complete | — |
| Injection Wrong | Injection Item Complete | — |

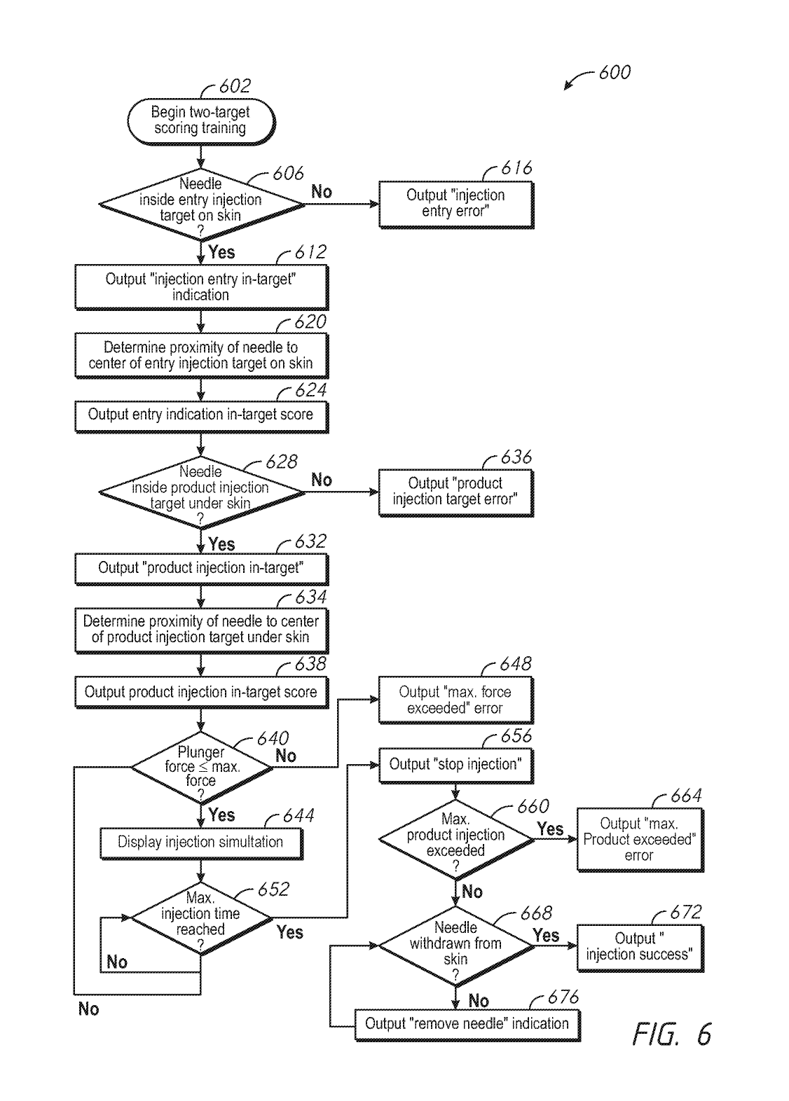

FIG. 6 is a process flow diagram for a method of providing a two-target scoring training 600. The two-target scoring training has one entry target on the skin of the training head and one product injection target underneath the skin. The method shown in FIG. 6 may be performed in one or more of the devices shown herein.

The method 600 begins at node 602 where a trainee begins the two-target scoring training. In some embodiments, the trainee begins the two-targeting scoring training by pushing a corresponding button on the user interface.

At decision node 606, the system determines if the syringe needle is within an injection entry target. If the needle is outside the injection entry target as determined by the sensors in the training head, the system can output and display on the user interface an “Injection Entry Error” message or an equivalent indication, such as shown in FIGS. 4A-C at node 616. If the needle is within the target, at node 612, the system can output and display on the user interface an “Injection Entry In-Target” message or an equivalent indication, such as shown in FIGS. 4A-C. At node 620, the system can optionally determine how close the needle is to the center of the target. At node 624, the system can optionally output and display an injection entry in-target score as described above.

Next, at decision node 628, the system can determine if the needle is now within a product injection target. Similar to the node 616, if the needle is not within the product injection target, the system can output and display on the user interface a “Product Injection Target Error” message or an equivalent indication at node 636. If the needle is within the target, at node 632, the system can output and display on the user interface a “Product Injection In-Target” message or an equivalent indication. At node 634, the system can optionally determine how close the needle is to the center of the product injection target. At node 638, the system can optionally output and display a product injection in-target score as described above.

Once the needle is within both the injection entry and product injection targets, at decision node 640, the system can monitor continuously or periodically, such as every 0.5 second, whether the plunger force as measured by the plunger force sensor on the syringe has exceeded the maximum injection force. If the maximum injection force is exceeded, the system can output and display a “Max. Force Exceeded” or equivalent error message at node 648. If the maximum force is not exceeded, the system can continue to display a simulation of the injection procedure at node 644. The system can determine at decision node 652 if a maximum injection time has been reached. If the maximum injection time has not been reached, the system can loop back to the decision node 640 to continue monitoring the plunger force. If the maximum injection time has been reached, the system can output a “Stop Injection” or equivalent message at node 656. The system can optionally determine at decision node 660 if the maximum product injection volume has been exceeded. In the illustrated embodiment, the system can calculate the amount of product injected into the training head by multiplying the injection speed based on the plunger force information and the injection time. In other embodiments, the system can employ different methods known in the art for calculating the amount of product injected. If the amount of product injected exceeds the maximum injection volume, the system can output a “Max. Product Exceeded” or equivalent error message at node 664. If the amount of product injected does not exceed the maximum injection volume, the system can determine at decision node 668 if the needle has been withdrawn from the skin. If the needle tip is still detectable by the image sensors inside the training head, the system can output a “Remove Needle” instruction at node 676 and loop back to decision node 668 until the needle is withdrawn. Once the needle is withdrawn from the skin, the system can output an “Injection Success” or equivalent message at node 672.

Example steps of the two-target scoring training are provided in Table 2 below.

TABLE 3

| State Name | Action | Next State |
|---|---|---|
| SyringeWaiting | Syringe becomes visible to sensor, is inside | InjectionStarted |
| | of injection target area | |
| SyringeWaiting | Syringe enters skin, is visible outside of | InjectionWrongEntry |
| | injection target area | |
| InjectionStarted | Plunger is within product injection target area | ProductInjectStart |
| | and is depressed, but is below max force | |
| InjectionStarted | Plunger is within target area, and is depressed | ProjectInjectMaxError |
| | above max force | |
| InjectionStarted | Plunger is depressed, but syringe is not within | ProductInjectTargetError |
| | product injection target area | |
| ProductInjectStart | Plunger is depressed below max force and | ProductInjectStart |
| | time max IS NOT reached for that force | |
| | (measure force × time = max medication) | |
| ProductInjectStart | Plunger is depressed below max force and | ProductInjectComplete |
| | time max IS reached for that force (measure | |
| | force × time = max medication) | |
| | note that force × time visual is shown on | |
| | training page to assist doctor | |
| ProductInjectComplete | Syringe is withdrawn from skin. | InjectionConect |
| ProductInjectComplete | Plunger is depressed below max force and | ProductInjectExceededError |
| | force × time max IS EXCEEDED by allowable | |
| | amount for injection (measure force × time = | |
| | max medication) | |

The two-target scoring training can provide visual guides of both the entry location on the skin and the destination location for the needle under the skin. The two-target scoring training also provides training of the injection pressure and duration. By practicing hitting both targets and staying within the desired injection force and duration, the trainee can gradually develop muscle memory for, among other things, the desired angle and depth of needle entry, and/or the injection pressure and duration. In one embodiment, the two-target scoring method described herein can be repeated for training of the serial puncture technique.

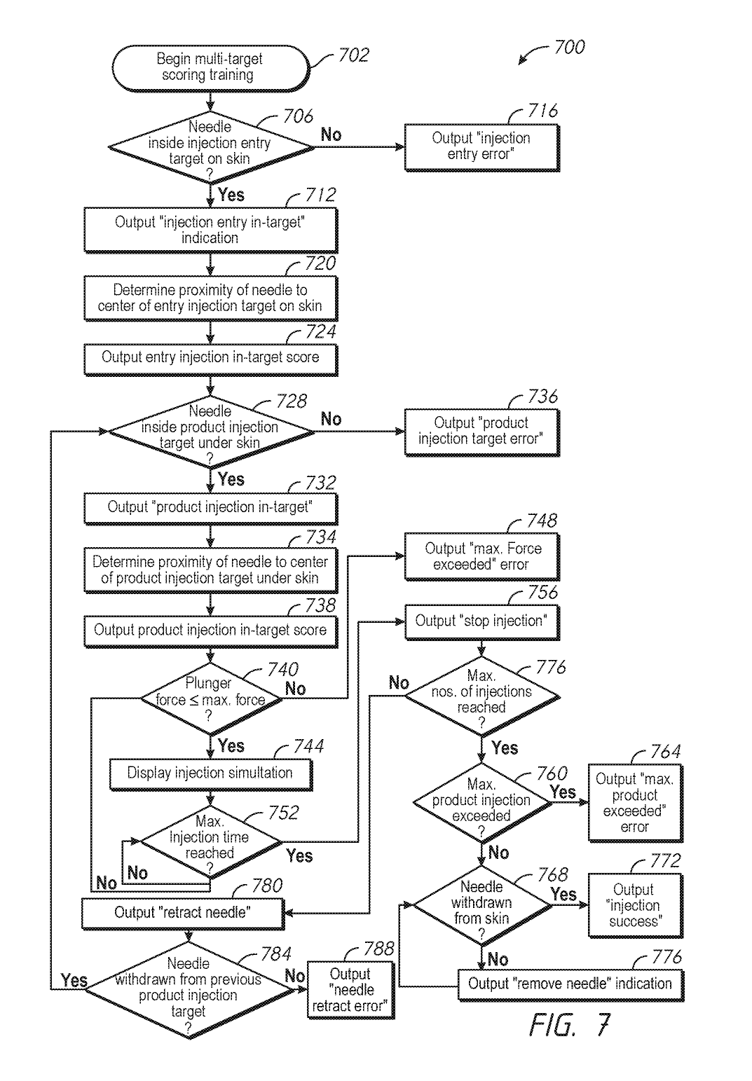

The system can also provide training with a multi-target scoring method, such as the method illustrated in FIG. 7. In some embodiments, the multi-target scoring method includes a single injection entry target and a plurality of product injection targets. In other embodiments, the multi-target scoring method includes a plurality of injection entry targets and a plurality of product injection targets. In some embodiments, the trainee performs one product injection after the syringe needle passes each injection entry target. In some embodiments, the trainee performs a plurality of product injections after the syringe needle passes each injection entry target. One of skill would recognize that the process flows illustrated in FIGS. 6-7 and described herein, individually or in various combinations, can be used for any multi-target scoring training. These multi-target techniques include but are not limited to cross hatching, serial puncture, serial threading, depot injection, Fern Pattern, cone, Z-Track method, and the like. FIG. 7 is a process flow diagram for a method of providing a multi-target scoring technique training 700. The multi-target scoring training uses a plurality of targets. One target can be an entry target on the skin of the training head. The other targets can be a plurality of product injection targets underneath the skin. For some multi-target techniques, the needle only penetrates the skin once and then moves to a new product injection target after having performed an injection at a current product injection target. For example, the fanning technique involves the needle sweeping underneath the skin between product injection targets. The linear threading technique involves the needle gradually retracting from the deepest product injection target to the product injection target closest to the entry target on skin. The method shown in FIG. 7 may be performed in one or more of the devices shown herein.

The method 700 begins at node 702 where a trainee begins the multi-target scoring technique training. In some embodiments, the trainee begins the fanning/linear threading technique training by pushing a corresponding button on the user interface. In some embodiments, the trainee makes a further selection between fanning and linear threading techniques for training.

At decision node 706, the system determines if the syringe needle is within an injection entry target. If the needle is outside the injection entry target as determined by the sensors in the training head, the system can output and display on the user interface an “Injection Entry Error” message or an equivalent indication, such as shown in FIGS. 4A-C at node 716. If the needle is within the injection entry target, at node 712, the system can output and display on the user interface an “Injection Entry In-Target” message or an equivalent indication, such as shown in FIGS. 4A-C At node 720, the system can optionally determine how close the needle is to the center of the target. At node 724, the system can optionally output and display an injection entry in-target score as described above.

Next, at decision node 728, the system can determine if the needle is now within a first product injection target. Similar to the node 716, if the needle is not within the product injection target, the system can output and display on the user interface an “Product Injection Target Error” message or an equivalent indication as shown in FIGS. 4A-C at node 736. If the needle is within the first product injection target, at node 732, the system can output and display on the user interface an “Product Injection In-Target” message or an equivalent indication, such as shown in FIGS. 4A-C. At node 734, the system can optionally determine how close the needle is to the center of the first product injection target. At node 738, the system can optionally output and display a product injection in-target score.

Once the needle is within both the injection entry and first product injection targets, at decision node 740, the system can monitor continuously or periodically, such as every 0.5 second, whether the plunger force has exceeded the maximum injection force. If the maximum injection force is exceeded, the system can output and display a “Max. Force Exceeded” or equivalent error message/indication at node 748. If the maximum force is not exceeded, the system can continue to display a simulation of the injection procedure as described above at node 744. The system can further determine at decision node 752 if a maximum injection time has been reached. If the maximum injection time has not been reached the system can loop back to decision node 740 to continue monitoring the plunger force. If the maximum injection time has been reached, the system can output a “Stop Injection” or equivalent message/indication at node 756.

At decision node 776, the system can determine if the maximum number of injections have been reached. In some embodiments, the maximum number of injection in a single fanning or linear threading technique can be from 3 to 6 injections. If the maximum number of product injections has not been exceeded, the system can output a “Retract Needle” or equivalent instruction at node 780. In some embodiments, the system can then determine if the needle has been withdrawn to near the entry target and underneath the skin for the fanning technique training at decision node 784. In some embodiments, the system can determine at the decision node 784 if the needle has been removed from the previous product injection target and towards a next product injection target closer to the skin for the linear threading technique training. If the needle has not been withdrawn to the desired location, the system can output a “Needle Retract Error” or equivalent message at node 788. If the needle has been withdrawn to the desired location, the system can loop back to the decision node 728 to determine the needle location and plunger force at the next product injection target.

After a predetermined number of product injection targets have been reached and injections have been performed in those targets, the system can further determine at decision node 760 if the maximum product injection volume has been exceeded. In the illustrated embodiment, the system can calculate the amount of product injected into the training head by multiplying the injection speed based on the plunger force information and the injection time. In other embodiments, the system can employ different methods known in the art for calculating the amount of product injected. If the amount of product injected exceeds the maximum injection volume, the system can output a “Max. Product Exceeded” or equivalent error message/indication at node 764. If the amount of product injected does not exceed the maximum injection volume, the system can determine at decision node 768 if the needle has been withdrawn from the skin. If the needle tip is still detectable by the image sensors inside the training head, the system can output a “Remove Needle” instruction at node 776 and loop back to decision node 768 until the needle is withdrawn. Once the needle is withdrawn from the skin, the system can output an “Injection Success” or equivalent message/indication at node 772.

Example steps of the multi-target scoring training are provided in Table 3 below.

| State Name | Action | Next State |
|---|---|---|
| SyringeWaiting | Syringe becomes visible to sensor, is inside of | InjectionStarted |
| | injection target area. | |
| SyringeWaiting | Syringe enters skin, is visible outside of | InjectionWrongEntry |
| | injection target area. | |
| InjectionStarted | Plunger is with first product injection target | ProductInjectStart |
| | area and is depressed, but is below max force. | |
| InjectionStarted | Plunger is within target area, and is depressed | ProjectInjectMaxError |
| | above max force. | (increment number of injections |
| | | effored) |
| InjectionStarted | Plunger is depressed, but syringe is not within | ProductInjectTargetError |
| | product injection target area. | (increment number of injections |
| | | effored) |
| ProductInjectStart | Plunger is depressed below max force and time | ProductInjectStart |
| | max IS NOT reached for that force (measure | |
| | force × time = max medication). | |
| ProductInjectStart | Plunger is depressed below max force and time | ProductInjectComplete |
| | max IS reached for that force (measure force × | |
| | time = max medication) | |
| | note that force × time visual is shown on | |
| | training page to assist doctor | |
| ProductInjectComplete | Increment number of injections completed. | NextInjection |
| | Syringe is withdrawn to skin target sphere area | |
| | but stays below skin. (# injections completed or | |
| | effored is less than max number of injection | |
| | sites) | |
| ProductInjectComplete | Number of injections completed or effored is | FanningInjectionsComplete |
| | less than max number of injection sites. | |
| NextInjection | Syringe moves outside skin target sphere area | InjectionStarted (loops until |
| | and into other area, below skin. (# injections is | injections complete) |
| | less than max number of injection sites) | |
| ProductInjectComplete | Syringe is withdrawn from skin. | InjectionConect |
| ProductInjectComplete | Plunger is depressed below max force and | ProductInjectExceededError |
| | force × time max IS EXCEEDED by allowable | (increment number of injections |
| | amount for injection (measure force × time = | effored) |
| | max medication). | |

The word “exemplary” is used herein to mean “serving as an example, instance, or illustration.” Any embodiment described herein as “exemplary” is not necessarily to be construed as preferred or advantageous over other embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein, one skilled in the art may appreciate that the scope of the disclosure is intended to cover any aspect of the novel systems, apparatuses, and methods disclosed herein, whether implemented independently of, or combined with, any other aspect described. For example, an apparatus may be implemented or a method may be practiced using any number of the aspects set forth herein. In addition, the scope of the described features is intended to cover such an apparatus or method which is practiced using other structure, functionality, or structure and functionality in addition to or other than the various aspects disclosed herein. It may be understood that any aspect disclosed herein may be embodied by one or more elements of a claim.

Although particular aspects are described herein, many variations and permutations of these aspects fall within the scope of the disclosure. Although some benefits and advantages of the preferred aspects are mentioned, the scope of the disclosure is not limited to particular benefits, uses, or objectives. Rather, aspects of the disclosure are broadly applicable to different injection training technologies, system configurations, networks, and transmission protocols, some of which are illustrated by way of example in the figures and the included description of the preferred aspects. The detailed description and drawings are merely illustrative of the disclosure rather than limiting, the scope of the disclosure being defined by the appended claims and equivalents thereof.

The terms “processor” and “processor module,” as used herein are a broad terms, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer. In some embodiments, the terms can include ROM and/or RAM associated therewith.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing, and the like.

As used herein, the term “message” encompasses a wide variety of formats for transmitting information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed/transmitted/stored/received/etc. in multiple parts.

Any reference to an element herein using a designation such as “first,” “second,” and so forth does not generally limit the quantity or order of those elements. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may include one or more elements.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such embodiment decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in an electronic device. In the alternative, the processor and the storage medium can reside as discrete components in an electronic device.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the disclosures described herein can be embodied within a form that does not provide all of the features and benefits set forth herein, as some features can be used or practiced separately from others. The scope of certain disclosures disclosed herein is indicated by the appended claims rather than by the foregoing description. All changes which come within the meaning and range of equivalency of the claims are to be embraced within their scope

1. An injection training system comprising:
a training apparatus configured to receive a training injection, the training apparatus comprising one or more training apparatus sensors;
a syringe configured to deliver the training injection, the syringe including a needle and one or more syringe sensors;
one or more processors in communication with the training apparatus and the syringe; and
a user interface in communication with the one or more processors;
wherein when a trainee performs the training injection on the training apparatus using the syringe, the one or more training apparatus and syringe sensors are configured to measure position and/or orientation information of the syringe and the training apparatus, and the one or more processors are configured to receive and process inputs from the one or more training apparatus and syringe sensors so as to output on the user interface three-dimensional digital models of the training apparatus and the syringe corresponding to the position and/or orientation of the syringe relative to the training apparatus as the trainee is performing the training injection.

2. The injection training system of claim 1, wherein the one or more training apparatus and syringe sensors are configured to measure one or more of injection force, injection duration, or injection volume, and the one or more processors are configured to receive and process inputs from the one or more training apparatus and syringe sensors so as to output feedback of the one or more of injection force, injection duration, or injection volume, and/or an outcome simulation.

3. The injection training system of claim 1, further comprising one or more training modules in communication with the one or more processors, the one or more training modules comprising instructions to a trainee performing the training injection, the instructions configured to be displayed on the user interface.

4. The injection training system of claim 3, wherein the one or more training modules comprise a two-target scoring training module, wherein the two-target scoring training module is configured to provide an injection entry target on a surface of the training apparatus and a product injection target under the surface of the training apparatus for display on the user interface.

5. The injection training system of claim 4, wherein the injection entry target and product injection target are configured to be displayed sequentially to trace movements of the needle during the training injection.

6. The injection training system of claim 3, wherein the one or more training modules comprise a multi-target scoring training module, wherein the multi-target scoring training module is configured to provide at least one injection entry target on a surface of the training apparatus and a plurality of product injection targets under the surface of the training apparatus for display on the user interface.

7. (canceled)

8. (canceled)

9. (canceled)

10. The injection training system of claim 1, wherein the one or more processors are configured evaluate the training injection based on the inputs from the one or more training apparatus and syringe sensors and evaluation logics from one or more training modules.

11. The injection training system of claim 1, wherein the one or more training apparatus sensors comprise an optical sensor.

12. The injection training system of claim 1, wherein the one or more syringe sensors comprises one or more of a 9-axis inertial motion sensor or a force sensor configured to measure injection force.

13. (canceled)

14. The injection training system of claim 1, wherein the syringe comprises a light emission source.

15. (canceled)

16. (canceled)

17. (canceled)

18. An injection training system for an injection technique comprising:
one or more processors in communication with and receiving inputs from one or more sensors on a training apparatus and/or on a needle; and
a user interface in communication with the one or more processors and configured to provide injection instructions associated with the injection technique, the injection instructions including:
an injection entry target on the training apparatus where the needle should penetrate the training apparatus, and
a product injection target on the training apparatus where the training injection should be delivered,
wherein the inputs from the one or more sensors on the training apparatus and/or needle comprise injection information associated with the training injection, the injection information including:
a needle entry location for the training injection, and
a product injection location where the training injection was delivered, and
wherein the user interface is configured to display indications of positions and/or orientations of the needle and training apparatus, and the one or more processors are configured to analyze the injection information including determining whether the training injection was delivered to the injection entry target and product injection target.

19. The system of claim 18, wherein the inputs from the one or more sensors on the training apparatus and/or needle further comprise one or more of an injection force or injection duration.

20. The system of claim 19, wherein the one or more processors are configured to evaluate the injection information relative to one or more of the following evaluation criteria:
a targeting accuracy score;
a depth of the training injection;
a duration of the training injection;
an angle of entry of the training injection;
an injection force; or
an amount of therapeutic agent delivered by the training injection.

21. An injection training system for an injection technique comprising:
one or more processors in communication with and receiving inputs from one or more sensors on a training apparatus and/or on a needle; and
a user interface in communication with the one or more processors and configured to provide injection instructions associated with the injection technique, the injection instructions including:
an injection entry target on the training apparatus where the needle should penetrate the training apparatus, and
a plurality of product injection targets on the training apparatus where a plurality of injections should be delivered after the needle penetrates the training apparatus,
wherein the inputs from the one or more sensors on the training apparatus and/or needle comprise injection information associated with the training injection, the injection information including:
a needle entry location for the training injection, and
a plurality of product injection locations on the training apparatus where the plurality of injections were delivered during the training injection, and
wherein the user interface is configured to display indications of positions and/or orientations of the needle and training apparatus, and the one or more processors are configured to analyze the injection information including determining whether the training injection was delivered to the injection entry target and plurality of product injection targets.

22. The system of claim 21, wherein the injection instructions comprise a plurality of injection entry targets and the injection information comprises a plurality of needle entry locations.

23. The system of claim 21, wherein the plurality of product injection targets project outward from the injection entry target and the injection instructions include a needle retracting target near the injection entry target under a surface of the training apparatus after each of the plurality of injections.

24. The system of claim 21, wherein the plurality of product injection targets are substantially linearly spaced from the injection entry target and from one another, and the training instructions include displaying sequentially the plurality of product injection targets with decreasing distance from the injection entry target.

25. The system of claim 21, wherein the inputs from the one or more sensors on the training apparatus and/or needle further comprise one or more of an injection force or injection duration.

26. The system of claim 25, wherein the one or more processors are configured to evaluate the injection information relative to one or more of the following evaluation criteria:
a targeting accuracy score;
a depth of the training injection;
a duration of the training injection;
an angle of entry of the training injection;
an injection force;
a maximum number of injections during the training injection; or
an amount of therapeutic agent delivered by the training injection.

27. The system of claim 21, wherein the training technique is one of fanning, linear threading, cross hatching, serial puncture, serial threading, depot injection, Fern Pattern, cone, or Z-Track method.

28.-34. (canceled)
